(19) United States
(12) Patent Application Publication
(10) Pub. No.: US 2022/0080084 A1
(43) Pub. Date: Mar. 17, 2022

(54) TISSUE PRODUCTS WITH ACTIVE AGENTS AND METHODS OF PRODUCTION

(71) Applicant: LifeCell Corporation, Madison, NJ (US)

(72) Inventors: Israel James Jessop, Garden Ridge, TX (US); Ming F. Pomerleau, Califon, NJ (US); Mrinal Shah, Parsippany, NJ (US); Ivis Munoz, East Stroudsburg, PA (US); Talia Connell, Manville, NJ (US)

(21) Appl. No.: 17/532,509

(22) Filed: Nov. 22, 2021

Related U.S. Application Data
(63) Continuation of application No. 16/254,145, filed on Jan. 22, 2019.
(60) Provisional application No. 62/620,716, filed on Jan. 23, 2018.

(51) Int. Cl. A61L 27/54; A61K 31/155; A61L 27/36

(52) U.S. Cl. CPC A61L 27/54; A61K 31/155; A61L 27/362; A61L 27/3691; A61L 27/36; A61L 2300/406; A61L 27/3629; A61L 27/3604; A61L 2300/404; A61L 2300/206; A61L 2300/408; A61L 27/3633

(57) ABSTRACT

The present disclosure relates to methods for producing tissue matrix products with active agents to achieve localized distribution and controlled active agent release. The method can include inserting a carrier element comprising a biodegradable material and active agent into surface features of a tissue matrix product. Also provided are tissue matrix products made using the disclosed methods.

This application is a continuation of U.S. application Ser. No. 16/254,145, filed Jan. 22, 2019, which claims priority under 35 U.S.C. § 119 to U.S. Provisional Application No. 62/620,716, which was filed on Jan. 23, 2018 and is herein incorporated by referenced in its entirety.

The present disclosure relates to tissue products, and more particularly, to tissue products with active agents, including methods of making such products.

Various tissue-derived products are used to regenerate tissue, facilitate wound healing, or otherwise treat diseased or damaged tissues and organs. Such products can include intact tissue grafts or acellular tissue matrices. For example, such tissue products may be provided in sheet form for use during soft tissue reconstruction surgery to connect tissues or to support implanted materials (e.g., hernia repair or breast support).

Although sheets of tissue matrix are valuable as tissue regeneration materials, it may be beneficial to improve upon the current tissue matrix compositions. For example, the addition of active agents, such as antimicrobial agents, to tissue matrices can prevent or treat infection or address other problems. Controlling the distribution, density, or elution rate associated with active agents in a tissue matrix product may provide further clinical benefits, and may result in improved patient outcomes.

The present application provides methods for producing tissue matrix products with active agents and products produced according to such methods. The disclosed methods can be used with active agents such as therapeutic agents.

### SUMMARY

According to various embodiments, a method of producing a tissue product is provided. The method may comprise selecting a tissue matrix and injecting active agent into the tissue matrix using at least one microneedle array at a first position on the tissue matrix. The active agent may comprise at least one of an antimicrobial, antibacterial, antifungal, antiviral, antiprotozoal, or antiseptic agent. In some embodiments, a tissue matrix product made using the disclosed methods is provided.

According to various embodiments, a method of producing a tissue product comprising selecting a tissue matrix and producing at least one surface feature in the tissue matrix is provided. The method can further comprise inserting a carrier element into the at least one tissue matrix surface feature. The carrier element comprises a biodegradable material and an active agent.

In some embodiments, a tissue matrix product is provided. The tissue matrix product may comprise a tissue matrix and tissue matrix surface features configured to receive a carrier element. The carrier element may comprise a biodegradable material and active agent. The tissue matrix surface features may comprise at least one of an indentation, groove, slot, or hole of various sizes and shapes. In some embodiments, the biodegradable material comprises one of a biodegradable liquid, biodegradable semi-solid, or biodegradable solid.

### BRIEF DESCRIPTION OF THE DRAWINGS

The present invention is illustrated by way of example, and not limitation, in the accompanying figures wherein:

FIG. 1A illustrates a tissue matrix and microneedle array for the production of tissue products, according to various embodiments of the present disclosure.

FIG. 1B illustrates a tissue matrix injected with active agent, according to various embodiments of the present disclosure.

FIG. 1C illustrates a microneedle array injecting active agent into a tissue matrix, according to various embodiments of the present disclosure.

FIG. 1D illustrates a microneedle array injecting active agent into a tissue matrix as it moves rearwardly/out of the tissue matrix, according to various embodiments of the present disclosure.

FIG. 2 illustrates a perspective view of tissue matrix injected with active agent, according to various embodiments of the present disclosure.

FIG. 3A illustrates a tissue matrix with carrier elements comprising an active agent, disposed within surface features of a tissue matrix, according to various embodiments of the present disclosure.

FIG. 3B illustrates an enlarged view of a section of tissue matrix from FIG. 3A, including a tissue matrix, surface feature, and a carrier element comprising a biodegradable material and active agent.

FIG. 4 illustrates a cross-sectional view of a tissue matrix on a cooling support surface as may be used in accordance with methods disclosed herein to produce surface features in tissue matrix.

FIG. 5A illustrates a cross-section view of a tissue matrix with randomly positioned surface features, according to various embodiments of the present disclosure.

FIG. 5B illustrates a perspective view of a tissue matrix with various types of slot surface features, according to various embodiments of the present disclosure.

FIG. 5C illustrates a cross-section view of a tissue matrix with hole surface features of various shapes and sizes, according to various embodiments of the present disclosure.

FIG. 6A illustrates a cross-section view of a tissue matrix with random surface features containing a carrier element, according to various embodiments of the present disclosure.

FIG. 6B illustrates a cross-section view of a tissue matrix with slot and hole surface features containing a carrier element, according to various embodiments of the present disclosure.

FIG. 6C illustrates a cross-section view of a tissue matrix with through-hole surface features containing a carrier element, according to various embodiments of the present disclosure.

FIG. 6D illustrates a cross-section view of a tissue matrix with pocket features containing a carrier element, according to various embodiments of the present disclosure.

FIG. 7 illustrates a method for treating a tissue matrix with active agents, according to various embodiments of the present disclosure.

### DESCRIPTION OF CERTAIN EXEMPLARY EMBODIMENTS

Reference will now be made in detail to various embodiments of the disclosed methods and devices, examples of which are illustrated in the accompanying drawings. Wherever possible, the same reference numbers will be used through the drawings to refer to the same or like parts. The drawings are not necessarily to scale.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

In this application, the use of the singular includes the plural unless specifically stated otherwise. Also in this application, the use of “or” means “and/or” unless stated otherwise. Furthermore, the use of the term “including,” as well as other forms, such as “includes” and “included,” are not limiting. Any range described here will be understood to include the endpoints and all values between the endpoints.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. To the extent publications and patents or patent applications incorporated by reference contradict the invention contained in the specification, the specification will supersede any contradictory material.

As used herein, “tissue product” will refer to any human or animal tissue that contains extracellular matrix proteins. “Tissue products” can include acellular or partially decellularized tissue matrices, as well as decellularized tissue matrices that have been repopulated with exogenous cells and/or cellular tissues.

As used herein “active agent” will refer to a substance or material that may be incorporated into a tissue product to improve overall clinical outcomes. “Active agents” may include therapeutic agents such as antiseptic, anti-inflammatory, antimicrobial, or angiogenic agents, among others.

As used herein “antimicrobial agent” will refer to a substance or material selected to kill or slow the growth of microorganisms. “Antimicrobial agents” may include antibacterial, antiviral, antifungal, or antiprotozoal agents. Various human and animal tissues can be used to make products for treating patients. For example, various tissue products for regeneration, repair, augmentation, reinforcement, and/or treatment of human tissues that have been damaged or lost due to various diseases and/or structural damage (e.g., from trauma, surgery, atrophy, and/or long-term wear and degeneration) have been produced. Such products can include, for example, acellular tissue matrices, processed tissue matrices (e.g., tissue matrices made into particulate, sponge-like, or composite forms), tissue allografts or xenografts, and/or reconstituted tissues (i.e., at least partially decellularized tissues that have been seeded with cells to produce viable materials).

For clinical applications, it is often desirable to provide tissue products that have certain mechanical and biologic properties. For example, certain tissue products may include a sheet of material, and may be implanted to repair defects like hernias, to support surrounding tissues or implants (e.g., for breast augmentation and/or reconstruction), or to replace damaged or lost tissue after trauma or surgical resection. Such tissue products should possess sufficient mechanical strength to withstand stresses and strains during their intended use until native tissue regeneration and repair is achieved.

Such tissue products may also benefit from the addition of certain active agents to further improve clinical outcomes. Such agents may be incorporated into tissue products disclosed herein to perform a variety of functions. For example, active agents may be provided to facilitate wound healing, promote native tissue ingrowth, increase vascularization, reduce inflammation, suppress negative immune response, prevent implant rejection, or a variety of other functions. The active agent may comprise at least one of a number of chemicals, for example, an anti-inflammatory, angiogenic protein, immunosuppressant, antibacterial, antifungal, antiviral, antiprotozoal, or antiseptic agent. In certain embodiments in which antimicrobial agents are the chosen active agent, exemplary classes of antimicrobial agents from which the active agent may be selected include, aminoclycosides, penicillins, cephalosporins, fluoroquinolones, glycopeptides, monobactams, carbapenems, and macrolides, among others.

In certain embodiments, active agents may comprise penicillin G, cephalothin, clavulanic acid, ampicillin, amoxicillin, methicillin, aztreonam, imipenem, rifampin, minocycline, streptomycin, gentamicin, vancomycin, clindamycin, erythromycin, polymyxin, bacitracin, amphotericin, rifampicin, tetracycline, ionic silver, silver oxide, silver nitrate, silver nanoparticle, poly(hexamethylene biguanide)hydrochloride (PHMB), chlorhexadine gluconate; bis-amido polybiguanides, honey, benzalkonium chloride, triclosan (2,4,4′-tricloro-2′-hydroxydiphenylether), and silyl quarternary ammonium salt (octadecyl demethyl trimethoxysilyl propyl ammonium chloride). It can be appreciated that a variety of active agents can be used in accordance with the devices and methods of the present disclosure. Additionally, in certain embodiments, one or more active agents may be provided in a variety of quantities and ratios in tissue products of the present disclosure.

The presently disclosed methods and devices can be used to process a variety of different tissues or tissue products. For example, the presently disclosed methods and devices can be used to incorporate active agents into any soft tissue or any tissue product derived from soft tissue. Such products derived from soft tissues include, for example, acellular tissue matrices, partially decellularized tissues, composite tissue matrices, reconstituted tissues, tissue allografts, autografts, or xenografts.

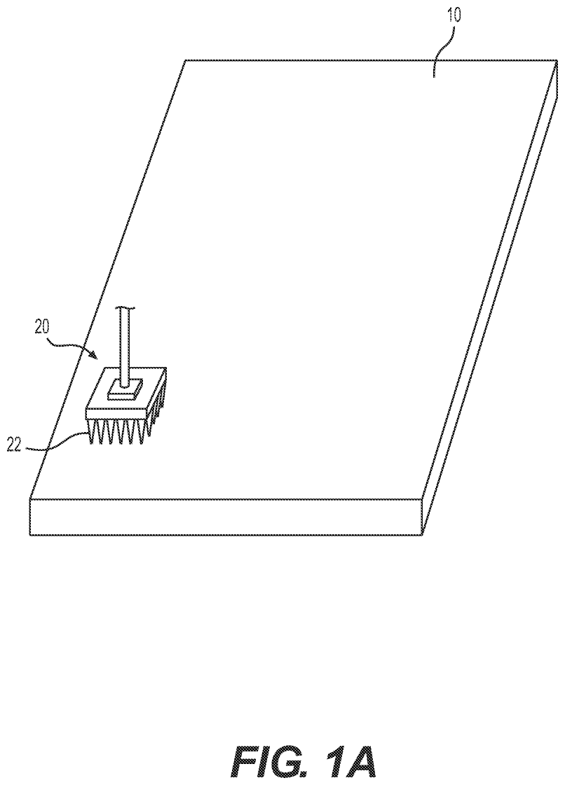

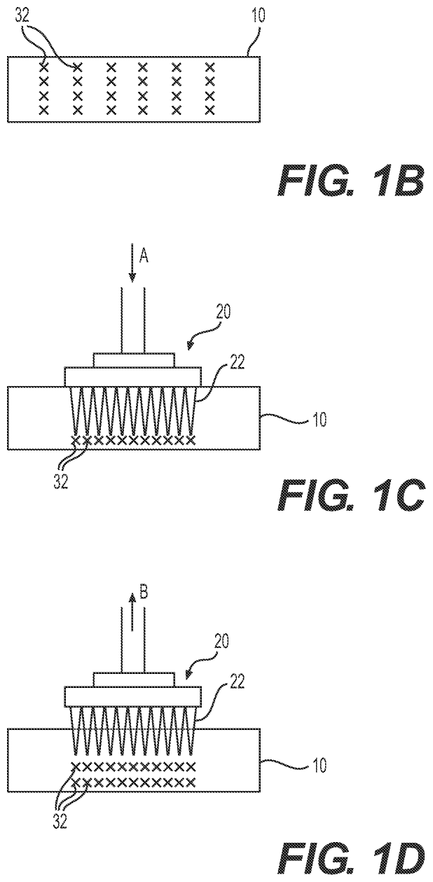

FIG. 1A illustrates tissue matrix 10 and microneedle array 20, comprising multiple microneedles 22, positioned to inject active agent into the tissue matrix. According to some embodiments, a method of producing a tissue product is disclosed, comprising selecting tissue matrix 10, and injecting active agent 32 (illustrated and discussed with respect to FIGS. 1B-1D) into tissue matrix 10 using at least one microneedle array 20 at a first position on tissue matrix 10. The microneedle array 20 may be connected to a container, such as a syringe (not pictured), that may house active agent 32. The container and microneedle array 20 may be in controlled, fluid communication to enable precise dispensation of active agent 32 within the tissue matrix 10.

FIGS. 1B-1D illustrate tissue matrix 10 containing active agent 32, and a process for injecting active agent 32 into tissue matrix 10, according to various embodiments of the present disclosure. FIG. 1B illustrates tissue matrix 10 injected with active agent 32, dispensed in a substantially uniform fashion, according to various embodiments. FIG. 1C illustrates microneedle array 20 injecting active agent 32 into tissue matrix 10. Microneedle array 20 has been moved from a position above tissue matrix 10 to a position within tissue matrix 10 along direction A, where direction A is oriented approximately perpendicular to the surface of tissue matrix 10. In FIG. 1C, the entire length or desired portion (depending on desired depth of injection) of microneedles 22 is penetrating tissue matrix 10. In certain embodiments, once microneedle array 20 has been inserted as depicted in FIG. 1C, microneedle array 20 may be activated to distribute active agent 32 from the tips of microneedles 22.

Microneedle array 20 can be produced from a variety of materials and may be provided in various configurations. In certain embodiments, microneedle array 20 is configured to be manipulated manually as to achieve a customizable distribution of active agent 32 within tissue matrix 10. In certain embodiments, microneedle array 20 may be connected to an automated system used to position and insert microneedle array 20 into tissue matrix 10. The automated embodiment of microneedle array 20 may be desirable for achieving precise control of the positioning of microneedle array 20 and the distribution of active agent 32. To dispense active agent 32 into a variety of tissue matrices 10, microneedle array 20 may be provided in various shapes and sizes.

Microneedle array 20 may be provided in a variety of shapes, including one of a circle, oval, triangle, rectangle, polygon, or various other forms. Similarly, microneedles 22 may be provided in various configurations, for example, conical, tetrahedral, cylindrical, or the like. Microneedles 22 may have lumen positioned along their lengths. In various embodiments, the lumen of microneedle 22 may be in fluid communication with a container used to store active agent 32. Microneedle array 20 may be configured to efficiently and precisely inject active agent 32 into tissue matrix 10. In certain embodiments, microneedle array 20 may be provided in a rectangular configuration with a uniform distribution of cylindrical microneedles 22 throughout the surface of microneedle array 20, comprising sharpened, beveled tips.

FIG. 1D illustrates microneedle array 20 injecting active agent 32 into tissue matrix 10 as microneedle array 20 moves rearwardly through or out of tissue matrix 10, according to various embodiments of the present disclosure. Microneedle array 20 may move rearwardly through tissue matrix 10 in direction B, where direction B is oriented approximately perpendicular to the surface of tissue matrix 10. As microneedle array 20 moves in direction B, active agent 32 is dispensed from the tips of microneedles 22. In various embodiments, the flow rate of active agent 32 and the retraction rate of microneedle array 20 may be controlled to provide a customized distribution of active agent 32 throughout tissue matrix 10.

In certain embodiments, flow rates of active agent 32 and retraction rates of microneedle array 20 may be adjusted to provide a variety of active agent 32 distribution throughout tissue matrix 10. For example, increasing flow rates of active agent 32 through microneedles 22 will result in increased amounts of active agent 32 per unit volume of tissue matrix 10. Retraction rates of microneedle array 20 will likewise impact density and distribution of active agent 32 throughout tissue matrix 10. For example, at a constant active agent 32 flow rate, faster retraction rates of microneedle array 20 will result in a lower density of active agent 32 throughout tissue matrix 10, as compared to slower retraction rates of microneedle 20. Various combinations of retraction rates of microneedle array 20 and flow rates of active agent 32 may be used with the disclosed devices and methods to yield tissue matrices 10 with varied or uniform distributions of active agent 32.

In some embodiments, the method of producing a tissue product may comprise positioning the at least one microneedle array 20 at a second position that is spatially distinct from, or partially overlaps, the first position on the tissue matrix 10, and injecting additional active agent 32 at the second position. Referring back to FIG. 1A, after microneedle array 20 has been inserted, active agent 32 injected, and microneedle array 20 removed from tissue matrix 10, microneedle array 20 may be positioned at a different location on tissue matrix 10 to inject, a second time, active agent 32. The method of producing a tissue product with active agent 32 may comprise repeatedly repositioning and inserting microneedle array 20, injecting active agent 32, and removing microneedle array 20 until the desired volume of tissue matrix 10 has been provided with active agent 32.

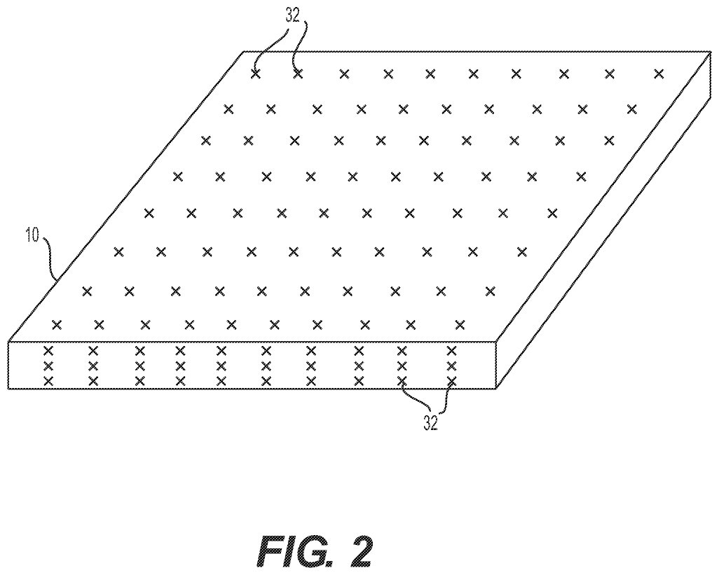

FIG. 2 illustrates a tissue matrix product comprising tissue matrix 10 and active agent 32, wherein the distribution and density of active agent 32 may be controlled using microneedle array 20, according to various embodiments of the present disclosure. In various embodiments, a tissue matrix product made by the disclosed methods is provided. Tissue matrix products provided with active agents may be used for regeneration, repair, augmentation, reinforcement, or treatment of human tissues that have been damaged or lost, for example, due to various diseases or structural damage. Such damage may be caused by, for example, trauma, surgery, atrophy, and/or long-term wear and degeneration. The disclosed tissue matrix products may include a sheet of material, and may be implanted in a patient to repair defects like hernias, to support surrounding tissues or implants (e.g., for breast augmentation and/or reconstruction), or to replace damaged or lost tissue after trauma or surgical resection.

In various embodiments, active agent 32 may be incorporated into tissue matrix 10 using a variety of other means. In certain embodiments, for example, a carrier element may be provided as a combination of a biodegradable, polymeric material and active agent 32. Instead of microneedle array 20 injecting active agent 32 into tissue matrix 10, naturally occurring or manufactured surface features of tissue matrix 10 may be used to carry a carrier element. As the carrier element degrades, active agent 32 may be released into the tissue matrix and treatment site. As such, a controllable dose of active agent 32 may be released within the body over time.

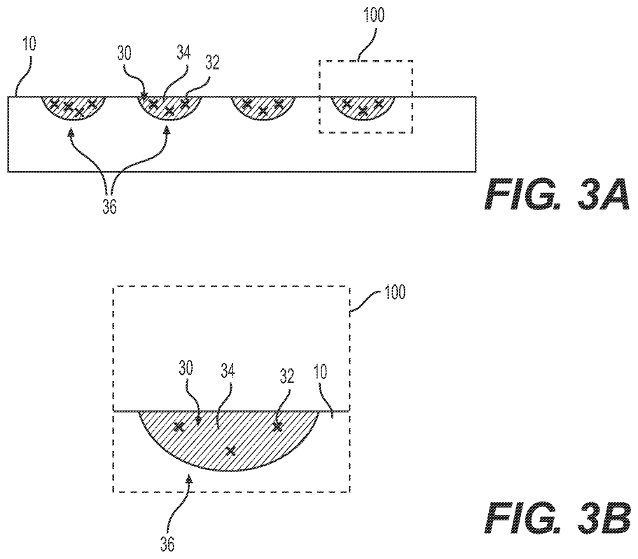

FIG. 3A illustrates a cross section of tissue matrix 10 with semi-spherical surface features 36 containing carrier element 30, according to various embodiments of the present disclosure. Carrier element 30 may comprise a biodegradable material 34 and active agent 32. In certain embodiments, a method of producing a tissue product is provided comprising selecting a tissue matrix 10, producing at least one surface feature 36 in tissue matrix 10, and inserting a carrier element 30 into the at least one surface feature 36. FIG. 3A also illustrates section 100. An enlarged rendering of section 100 is illustrated in FIG. 3B, which includes surface feature 36 in tissue matrix 10 containing carrier element 30.

Surface features 36 may be produced in tissue matrix 10 in a variety of different ways and may facilitate improved binding of carrier element 30 to tissue matrix 10. Surface features 36 may include increased surface roughness, geometric surface features, or partially swollen collagen on the surface of tissue matrix 10. Various chemical and mechanical processes may be used to produce surface features 36. Mechanical processes for producing surface features 36 may include, for example, cutting, scraping, stamping, ablating or various other techniques. Chemical modification techniques that remove or alter tissue, such as solvent annealing or colloidal lithography, among others, may be used to generate surface features 36 within or on tissue matrix 10.

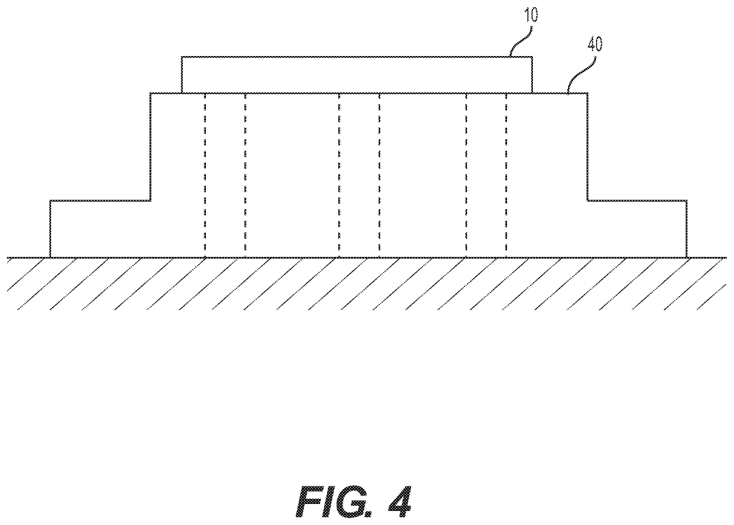

In certain embodiments, a mechanical process used to produce surface features 36 may include cooling tissue matrix 10 to enable stable and repeatable machining of tissue matrix 10. An example of a system for cooling tissue matrix 10 is provided in FIG. 4, illustrating a cross-sectional view of a tissue matrix 10 on a cooling support surface 40. At room temperature, tissue matrix 10 is typically provided in soft and malleable form. In various embodiments, the method of producing at least one surface feature 36 comprises a process including cooling tissue matrix 10 by contacting tissue matrix 10 with cooled support surface 40 wherein the cooled support surface 40 is cooled to stiffen tissue matrix 10. Various methods and techniques may be used to cool support surface 40 to sufficiently stiffen or rigidify tissue matrix 10. For example, cooled support surface 40 may be cooled by passing a cooling fluid near, or in contact with, the cooled support surface 40. Alternatively, a controlled release of liquid nitrogen or other coolant underneath or around support surface 40 may be used to cool support surface 40 and tissue matrix 10.

In certain embodiments, the disclosed method comprises cooling tissue matrix 10 to a temperature sufficient to enable machining of tissue matrix 10. The temperature to which tissue matrix 10 is cooled can be about 0, −5, −10, −15, −20, −25, −30, −35, −40, −45, −50, −55, −60, −65, −70, −75, −80, −85, −90, −95, −100, −105, −110, −115, −120, −125, −130, −135, or about −140° C. . These values may be used to define a single temperature, such as approximately −30° C. or −80° C. Alternatively, the listed temperature values may be used to define a range, such as −75 to −85° C., or −20 to −40° C. Exemplary temperatures and temperature ranges may generally result in tissue matrix 10 possessing sufficient rigidity to be machined, but do not result in a brittle tissue material that may shatter under high mechanical stresses caused by machining.

In certain embodiments, the tissue matrix 10 comprises surface features 36 configured to receive carrier element 30. Surface features 36 of the present disclosure may comprise at least one of an indentation, groove, slot, or hole of various size and shape extending partially or completely along one or more dimensions of the tissue matrix. The sizes, shapes, and quantities of surface features 36 can be configured to achieve desired clinical outcomes. For example, the volume of surface features 36 may be increased or decreased to, in turn, increase or decrease the volume of carrier element 30 contained therein. Additionally, the shapes and sizes of surface features 36 can be configured to expose larger or smaller areas of carrier element 30 to the body of a patient, which may influence the degradation rates of carrier element 30, and, concurrently, the elution rate of active agent 32.

In various embodiments, tissue matrix 10 may comprise surface features 36 positioned randomly, in a patterned configuration, or uniformly throughout tissue matrix 10. Configurations of surface features 36 may be altered for different applications. For example, in various embodiments of the present disclosure, where precise degradation of biodegradable material 34, and thus elution of active agent 32, is not required, random surface features may suffice. Relying on natural surface features to contain carrier element 30 may reduce overall production and manufacturing time of the disclosed tissue product. In various embodiments of the present disclosure where more precise elution or distribution of active agent 32 is desired, surface features 36 of tissue matrix 10 may be machined with high precision to produce uniform surface features 36 with precise volumes. In tissue products where surface features 36 are machined to tight tolerances, precise volumes or locations of carrier element 30 may be injected.

Surface feature patterns of the present disclosure may be selected based on implantation site or chosen treatment method. For example, in various embodiments, the surface feature pattern may be selected to provide a tissue matrix having improved mechanical properties. For example, in supporting implanted tissues in the chest, tissue matrix products may require high load-bearing capabilities in the horizontal direction, and increased flexibility in the transverse direction. To accommodate this mechanical performance requirement, one or more longitudinal slots may be machined into the sheet of tissue matrix. Longitudinal slots can provide increased flexibility along the horizontal axis to accommodate changing implant geometries. Malleable, biodegradable pastes 34 containing active agent 32 may be inserted into the longitudinal slots to reduce risk of microbial colonization, and implant rejection, while increasing flexibility along the horizontal axis of the sheet of tissue matrix 10.

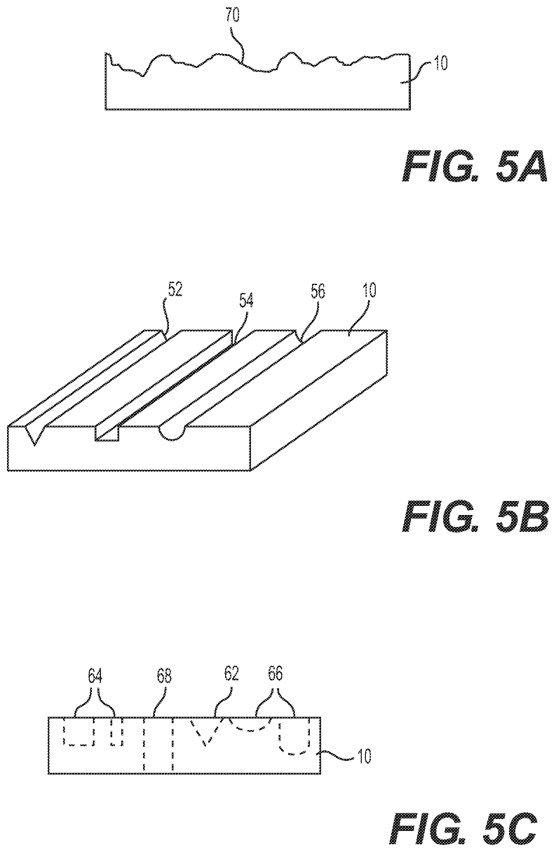

FIGS. 5A-5C illustrate various types of surface features 36 according to various embodiments of the present disclosure. For example, FIG. 5A illustrates a cross section of tissue matrix 10 with randomly or irregularly shaped and distributed surface features 70. Random or irregular surface features 70 may present naturally in tissue matrix, or may be produced. FIG. 5B illustrates a perspective view of tissue matrix 10 with various types of slot surface features comprising long apertures or grooves, extending along one direction of tissue matrix 10. The cross section of slot surface feature 52 comprises a triangle and may be machined in cooled tissue matrix 10 with a triangle-tipped cutting tool. The cross section of slot surface feature 54 comprises a rectangle and may be machined in cooled tissue matrix 10 with a rectangular-tipped cutting tool. The cross section of slot surface feature 56 comprises a semi-circle and may be machined in cooled tissue matrix 10 with a round-tipped cutting tool. In certain embodiments, slot surface features may be shallow or deep, depending on the desired volume of carrier element 30 to be inserted therein. To ensure the integrity and viability of the tissue matrix, in various embodiments, the slot surface features 52, 54, 56 of FIG. 5B do not extend through the thickness of tissue matrix 10.

FIG. 5C illustrates a cross-section view of tissue matrix 10 with hole surface features of various shapes and sizes according to various embodiments of the present disclosure. In certain embodiments, hole features may extend partially or completely through the tissue matrix. The size of the hole features determines the volume of carrier element 30 that can be inserted into tissue matrix 10. In certain embodiments, hole features 64 may have rectangular cross sections with varying dimensions. Hole feature 68 is a through-hole extending along the thickness of tissue matrix 10. Hole feature 62 has a triangular cross section. Hole features 66 have rounded bottoms and varying depths. Tissue matrix 10 may comprise one or more hole features. In certain embodiments, the hole features may be uniformly disposed throughout tissue matrix 10, or may vary in size, shape, and cross-sectional area throughout tissue matrix 10.

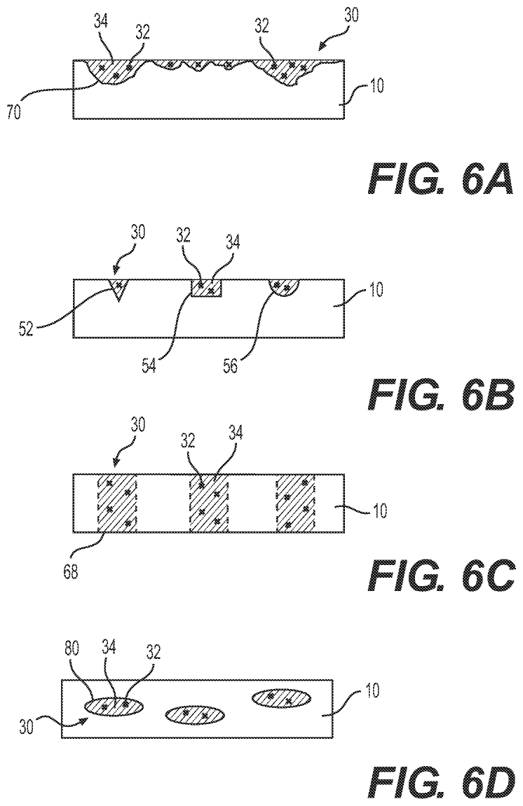

In various embodiments, carrier element 30 may bond to tissue matrix 10 using a variety of mechanisms. In some embodiments, carrier element 30 adheres to tissue matrix 10 via ionic, covalent, or hydrogen bonds. In certain embodiments, carrier element 30 may bond with tissue matrix 10 using mechanical means, such as press fit methods. Examples of carrier elements 30 bonded to tissue matrix 10 are illustrated in the accompanying FIGS. 6A-6D.

FIGS. 6A-6D illustrate tissue matrices 10 with varying surface features containing carrier elements 30, according to various embodiments of the present disclosure. FIG. 6A illustrates a cross-section view of tissue matrix 10 with random or irregular surface features 70. Within random or irregular surface features 70 lies carrier element 30, comprising active agent 32 and biodegradable material 34. To fill surface features 70, in certain embodiments, carrier element 30 may comprise a malleable, biodegradable paste 34. Biodegradable paste 34 may comprise a polymeric material that may degrade over time and provide a sustained release of active agent 32 within a treatment site. Alternatively, biodegradable material 34 may comprise biocomposites designed to be absorbed by the body of the patient, with customizable elution rates of active agent 32.

FIG. 6B illustrates a cross-section view of a tissue matrix 10 with slot and hole surface features containing a carrier element 30. In certain embodiments, features 52, 54, 56 are slots extending along a length or width dimension of tissue matrix 10. In certain embodiments, features 52, 54, 56 are holes existing at points along the top surface of tissue matrix 10. Features 52, 54, 56 comprise triangular, rectangular, and semi-circular cross sections, respectively. The sizes, shapes, and quantities of surface features in tissue matrix 10 can be configured to carry a desired volume of carrier element 30.

FIG. 6C illustrates a cross-section view of tissue matrix 10 with through-hole surface features 68 containing carrier element 30. Through-hole surface features 68 provide additional surface area (area where coating is directly exposed to the body) or volume compared to slot-type and hole-type surface features. Through-hole surface features 68 may have a variety of cross-sections, including circular, oval, rectangular, triangular, or polygonal. The cross-section of through-hole surface features 68 may be uniform throughout their length, or it may vary, depending on clinical need.

FIG. 6D illustrates a cross-section view of tissue matrix 10 with pocket features 80 containing carrier element 30, according to various embodiments of the present disclosure. As tissue matrix 10 may promote native tissue ingrowth and regeneration, pocket feature 80 may degrade from within tissue matrix 10 and slowly elute active agent 32 to a treatment site. In certain embodiments, biodegradable agent 34 may comprise, in part, a polymeric capsule that may have a slow degradation rate to facilitate elution of active agent 32 over an extended period of time.

In certain embodiments, carrier element 30 comprises a biodegradable liquid, biodegradable semi-solid, biodegradable solid material, or any combination therebetween. Carrier element 30 may be selected to achieve the desired clinical results. For example if carrier element 30 possesses a slow degradation rate, a slow delivery rate of active agent 32 to the treatment site will result. Alternatively, if carrier element 30 possesses an accelerated degradation rate, a fast delivery rate of active agent 32 to the treatment site will result. As such, the selection of biodegradable material 34 for the disclosed method may be highly variable, depending on the tissue product application.

As an example, in some embodiments, biodegradable material 34 may be selected from a number of polymer types. As used herein, the polymeric materials can include synthetic polymers and/or naturally occurring polymers. Further, the polymeric materials can include individual polymers and/or polymer mixtures (e.g., copolymers). In some embodiments, the polymeric materials can include polyglycolide, polylactide, polydioxane (or other polyether esters), poly(lactide-co-glycolide), and/or polyhydroxyalkonates. For example, in certain embodiments, the polymeric material can include polyhydroxyalkonates such as, for example, polyhydroxybutyrate (e.g., poly-3-hydroxybutyrate, poly-4-hydroxybutyrate (P4HB)), polyhydroxyvalerate, polyhydroxyhexanoate, polyhydroxyoctanoate, or trimethylene carbonate. Alternatively or additionally, the polymeric material can include polycaprolactone (PCL) and/or hyaluronic acid derivatives (e.g., esters, anhydrides, etc.), such as, for example, a benzyl ester derivative of hyaluronic acid (BHA). In various embodiments, biodegradable material 34 may be configured or processed to contain a uniform distribution of active agent 32 per unit volume of biodegradable material 34. Further, the selection of biodegradable material 34 may be such that it does not illicit a biologic response after implantation.

In certain embodiments of the present disclosure, the at least one polymeric material, selected to comprise biodegradable material 34, can provide structure to carrier element 30. The structure can increase biocompatibility and stability of carrier element 30 and may prevent migration of carrier element 30 from tissue matrix 10 or the treatment site. Accordingly, the distribution of active agent 32 can be controlled throughout tissue matrix 10. For example, in various embodiments, carrier element 30 may comprise a malleable polymeric paste or polymeric capsule. In various embodiments of the present disclosure, the need for biodegradable material 34 to carry active agent 32 is eliminated when a collagen scaffold is provided, manufactured using methods described in further detail below.

For certain clinical applications, it may be desirable to provide a low density tissue matrix 10 with active agent 32. Low density tissue matrices may be used for wound healing or other clinical applications. Accordingly, in certain embodiments, a method for producing a tissue product of the present disclosure comprising a low density, collagen scaffold is provided. The method may include grinding collagen to produce decellularized collagen fibers. Next, the ground, decellularized collagen fibers may be re-suspended in a suitable buffer, and active agent 32 may be added. In certain embodiments, the mixture of ground, decellularized collages fibers, buffer, and active agent 32 comprises a slurry.

The slurry may contain a suitable percentage of collagen fiber solids. For example, the percentage of collagen fiber solids may contain 0.1, 0.2, 0.3, 0.4, 0.5, 0.7, 1.0, 1.5, 2.0, 2.5, 3.0, 5.0, 6.0, 7.0, 8.0, 9.0, 10.0, 15.0, 20.0, 25.0, 30.0, 40.0, or 50.0% collagen fiber solids. These values may be used to define a range, such as 2-10% collagen fiber solids. Next, the slurry, comprising active agent 32 in the buffer, undergoes lyophilization, or freeze-drying. During lyophilization, moisture is removed from the product in a frozen state, leaving behind a porous, three-dimensional collagen structure with a uniform distribution of active agent 32.

Although there are various methods, and combinations of methods to make a tissue product of the present disclosure, any one of the multiple methods disclosed herein may be successfully implemented to add active agent 32 to a tissue product as is desired or best suited for the intended application of the tissue product.

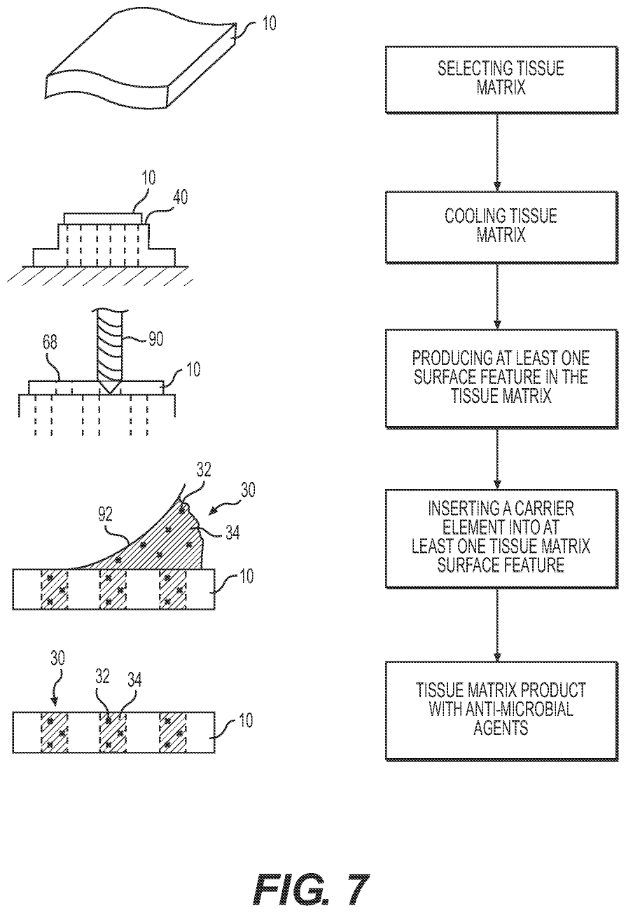

FIG. 7 illustrates a method for adding active agent 32 to tissue matrix 10, according to various embodiments of the present disclosure. The method begins with selecting tissue matrix 10, followed by cooling tissue matrix 10 on a cooling surface 40. Tissue matrix 10 is then machined by drill bit 90 to produce surface features 68. Surface features may be produced with other means, or may be present in the tissue, as described previously. Next, carrier element 30 is inserted into surface features 68 of tissue matrix 10, for example, using a spackling technique where carrier 30 comprises biodegradable paste 34 and active agent 32. In some embodiments, in forming carrier element 30, active agent 32 may be uniformly mixed into biodegradable paste 34. Carrier element 30 may be forced into surface features 68 using various means, including spackling blade 92. Tissue matrix 10 with carrier element 30 and active agent 32 is displayed, and may then be sterilized using a variety of suitable methods. For example, the tissue matrix 10 with active agent 32 may be sterilized using e-beam sterilization.

The tissue products disclosed herein can be made from a variety of suitable tissue sources. Examples of the tissues that may be used to construct the tissue matrices for the first component can include, but are not limited to, skin, parts of skin (e.g., dermis), fascia, muscle (striated, smooth, or cardiac), pericardial tissue, dura, umbilical cord tissue, placental tissue, cardiac valve tissue, ligament tissue, tendon tissue, blood vessel tissue, such as arterial and venous tissue, cartilage, bone, neural connective tissue, urinary bladder tissue, ureter tissue, and intestinal tissue. For example, a number of biological scaffold materials that may be used for tissue matrix 10 are described by Badylak et al., Badylak et al., “Extracellular Matrix as a Biological Scaffold Material: Structure and Function,” Acta Biomaterialia (2008), doi:10.1016/j.actbio.2008.09.013. In some cases, tissue matrix 10 includes a sheet of acellular tissue matrix derived from human or porcine dermis. Suitable human and porcine dermal materials include, for example, ALLODERM® and STRATTICE™, respectively.

Tissue matrices 10 may be processed in a variety of ways, as described below, to produce decellularized or partially decellularized tissues. In general, the steps involved in the production of an acellular tissue matrix 10 include harvesting the tissue from a donor and cell removal under conditions that preserve biological and structural function. In certain embodiments, the process includes chemical treatment to stabilize the tissue and avoid biochemical and structural degradation together with or before cell removal. In various embodiments, the stabilizing solution arrests and prevents osmotic, hypoxic, autolytic, and proteolytic degradation, protects against microbial contamination, and reduces mechanical damage that can occur with tissues that contain, for example, smooth muscle components. The stabilizing solution may contain an appropriate buffer, one or more antioxidants, one or more oncotic agents, one or more antibiotics, one or more protease inhibitors, and/or one or more smooth muscle relaxants.

The tissue may then be placed in a decellularization solution to remove viable cells, which include epithelial cells, endothelial cells, smooth muscle cells, and fibroblasts, from the tissue matrix without damaging the biological and structural integrity of the collagen matrix. The decellularization solution may contain an appropriate buffer, salt, an antibiotic, one or more detergents, one or more agents to prevent cross-linking, one or more protease inhibitors, and/or one or more enzymes. In some embodiments, the tissue is incubated in the decellularization solution overnight. In certain embodiments, additional detergents may be used to remove fat from the tissue sample.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A tissue product prepared by a process, comprising:
selecting an acellular tissue matrix;
positioning at least one microneedle array at a desired depth within the acellular tissue matrix; and
injecting an active agent into the acellular tissue matrix using the at least one microneedle array at a first position on the tissue matrix while controlling the injection rate and retraction rate of the microneedle array to produce a desired distribution of the active agent through a thickness of the acellular tissue matrix.

2. The tissue product of claim 1, wherein the process further comprises positioning the at least one microneedle array at a second position that is spatially distinct from the first position on the acellular tissue matrix and injecting additional active agent at the second position.

3. The tissue product of claim 2, further comprising repeatedly positioning the at least one microneedle array at least one additional position that is spatially distinct from previous injection positions on the acellular tissue matrix and injecting additional active agent.

4. The tissue product of claim 1, wherein the active agent includes antimicrobial agents, comprising at least one of an antibacterial, an antifungal, an antiviral, or an antiprotozoal agent.

5. The tissue product of claim 4, wherein the antimicrobial agent comprises chlorhexidine digluconate.

6. The tissue product of claim 1, wherein the process further comprises sterilizing the acellular tissue matrix.

7. A tissue matrix product comprising:
a tissue matrix;
tissue matrix surface features configured to receive a carrier element; and
a carrier element comprising a biodegradable material and active agents.

8. The tissue matrix product of claim 7, wherein the tissue matrix comprises a sheet.

9. The tissue matrix product of claim 7, wherein the tissue matrix comprises a product derived from at least one of adipose tissue, dermis, muscle, pericardium, nerve tissue, intestinal tissue, bladder, stomach, fascia, tendon, ligament, lung, liver, pancreas, or kidney.

10. The tissue matrix product of claim 7, wherein the tissue matrix comprises a product derived from mammalian dermis.

11. The tissue matrix product of claim 7, wherein the tissue matrix comprises a decellularized tissue matrix.

12. The tissue matrix product of claim 7 wherein tissue matrix surface features configured to receive a carrier element comprise at least one of an indentation, groove, slot, or hole of various size and shape extending partially or completely along one or more dimensions of the tissue matrix.

13. The tissue matrix product of claim 7, wherein the at least one surface feature is positioned randomly, in a patterned configuration, or uniformly throughout the tissue matrix.

14. The tissue matrix product of claim 7, wherein the carrier element adheres to the tissue matrix via ionic, covalent, or hydrogen bonds.

15. The tissue matrix product of claim 7, wherein the carrier element comprises at least one of a biodegradable liquid, biodegradable semi-solid, biodegradable solid material, or any combination thereof.

16. The tissue matrix product of claim 15, wherein the biodegradable material comprises at least one of a malleable paste, polymeric capsule, collagen scaffold, or any combination thereof.

17. The tissue matrix product of claim 16, wherein the biodegradable material does not illicit an excessive inflammatory response.

18. The tissue matrix product of claim 7, wherein the active agent comprises at least one of an antimicrobial, antibacterial, antifungal, antiviral, antiprotozoal agent, or antiseptic material.

19. The tissue matrix product of claim 18, wherein the antimicrobial agent comprises chlorohexidine digluconate.

20. The tissue matrix product of claim 7, wherein the tissue matrix is aseptic or sterile.
